(19) United States
(12) Patent Application Publication
(10) Pub. No.: US 2003/0157539 A1
(43) Pub. Date: Aug. 21, 2003

(54) IRAK-M is a negative regulator of toll-like receptor signaling

(75) Inventors: Richard A. Flavell, Guilford, CT (US); Koichi Kobayashi, Branford, CT (US); Ruslan M. Medzhitov, Branford, CT (US)

Correspondence Address: ROPES & GRAY LLP, ONE INTERNATIONAL PLACE, BOSTON, MA, 02110-2624, US

(73) Assignee: Yale University, New Haven, CT

(21) Appl. No.: 10/340,545

(22) Filed: Jan. 9, 2003

Related U.S. Application Data
(60) Provisional application No. 60/348,176, filed on Jan. 9, 2002.

(51) Int. Cl. C12Q 1/68; C07H 21/04; C12P 21/02; C12N 5/06; C07K 14/705

(52) U.S. Cl. 435/6; 435/69.1; 435/320.1; 435/325; 530/350; 536/23.2

(57) ABSTRACT

Isolated nucleic acid encoding the amino acid sequence of murine IRAK-M; expression vectors comprising nucleic acid encoding IRAK-M and host cells comprising them are disclosed. IRAK-M is induced upon toll-like receptor (TLR) stimulation and negatively regulates TLR signaling. Methods for identifying antagonists and agonists of IRAK-M are described.

### RELATED APPLICATION

This application claims the benefit of the filing date of U.S. Provisional Application No. 60/348,176, filed Jan. 9, 2002 and entitled “IRAK-M is a negative regulator of toll-like receptor signaling” by Richard A. Flavell, Koichi Kobayashi and Ruslan Medzhitov. The entire teachings of the referenced provisional application are incorporated herein by reference.

### FUNDING

Work described herein was supported by National Institutes of Health Grant number P01 AI 36529. The United States Government has rights in the invention.

### BACKGROUND OF THE INVENTION

Toll-like receptors (TLRs) provide an evolutionarily conserved detection system to recognize microorganisms and protect multicellular organisms from infection. A better understanding of immune system regulation would provide opportunities to develop approaches to modulating immune responses.

### SUMMARY OF THE INVENTION

The present invention relates to isolated IRAK-M protein, such as mouse IRAK-M protein; nucleic acids (DNA, RNA) encoding IRAK-M protein, such as mouse nucleic acids; expression vectors comprising nucleic acids encoding IRAK-M proteins; host cells containing such expression vectors; cells that are IRAK-M deficient, such as cells (e.g., mouse, human cells) that do not comprise nucleic acids that encode functional TRAK-M and IRAK-M⁻¹⁻ cells and methods of producing IRAK-M, such as mouse IRAK-M. It further relates to methods of identifying compounds that modulate the innate immune response in an individual, comprising combining or contacting cells expressing IRAK-M with a candidate compound and determining whether the candidate compound modulates IRAK-M activity in the cells. Modulation of IRAK-M activity in the cells by the candidate compound indicates that the candidate compound modulates the innate immune response in the individual. In one embodiment, the cells and the candidate compound are combined (contacted) under conditions appropriate for entry of the candidate compound into the cells. In one embodiment, the invention is a method of identifying compounds that enhance the innate immune response by inhibiting IRAK-M activity in cells. In this embodiment, the method further comprises the step of comparing IRAK-M activity in cells in the presence of the candidate compound with IRAK-M activity of a standard known to be deficient in IRAK-M activity. IRAK-M activity in the presence of the candidate compound comparable to IRAK-M activity for the standard indicates that the candidate compound is an IRAK-M inhibitor and one that enhances the innate immune response (e.g., production of inflammatory cytokines or chemokines). In one embodiment, the method of identifying compounds is carried out in cells which do not express IRAK-M. A further embodiment of the present invention is a method of identifying a compound that produces an anti-inflammatory effect and an immunoinhibitory effect in a subject, comprising combining or contacting cells that express IRAK-M with a candidate compound and determining whether the candidate compound enhances IRAK-M activity in the cells, wherein if enhancement of IRAK-M activity occurs in the cells, a compound that produces an anti-inflammatory effect and an immunoinhibitory effect is identified. In another embodiment, the present invention is a method of treating an inflammatory condition in a subject (individual) comprising administering to the subject a compound that enhances IRAK-M activity in cells in the subject, thereby producing an anti-inflammatory effect in the subject. The method of treatment can be used to treat a variety of inflammatory conditions, such as an autoimmune condition (e.g., rheumatoid arthritis, lupus erythematosis).

TLRs transduce their signals through downstream adapter molecules, MyD88 and the serine/threonine Kinase IRAK. The IRAK family consists of three proteins, IRAK and the inactive Kinases IRAK2 and IRAK-M. Here we show that IRAK-M is induced upon TLR stimulation and negatively regulates TLR signaling. IRAK-M deficient cells exhibited increased cytokine production upon TLR stimulation and bacterial challenge, and IRAK-M deficient mice showed increased inflammatory responses to bacterial infection. Endotoxin tolerance, a protection mechanism against endotoxin shock, was significantly reduced in IRAK-M deficient cells. Retroviral transduction into IRAK-M deficient cells of IRAK-M, IRAK2 and IRAK mutated in the Kinase domain, but not wild-type IRAK reduced cytokine production upon TLR stimulation. As described herein, IRAK-M is a critical regulator in TLR signaling and essential for the maintenance of the homeostasis of the innate immune system.

### BRIEF DESCRIPTION OF THE DRAWINGS

FIGS. 1A-1D: Molecular cloning and Targeted Disruption of the Mouse irak-M Gene.

FIG. 1A: Schematic representation of the Kinase domain of mouse IRAK-M and other Pelle/IRAK family proteins. The conserved motif and the amino acid sequence of mouse IRAK-M, human IRAK-M (accession number AF113136), human IRAK (accession number L76191), human IRAK2 (accession number AF(026273) and Drosophila Pelle (L08476) are shown. The conserved lysine on ATP binding site in subdomain II and the catalytically active aspartate are highlighted with shading. The entire sequence of mouse IRAK-M cDNA was submitted to GenBank (accession number AF461763).

FIG. 1B: Schematic diagram of the mouse irak-M gene locus, the targeting vector and the targeted allele. Filled boxes donate the coding exons. Restriction enzyme sites are indicated (S, Sph I; EV, EcoR V; X, Xba I, A, Apa I; B, BamH I). The probe used for the genotyping of the mutant mice was indicated by a bar.

FIG. 1C: Targeted disruption of the mouse irak-M gene. Southern blot analysis of genomic DNA identifies mice corresponding to the expected genotypes. Sph I digested DNA was probed as indicated. The upper band (6.3 kb) corresponds to the wild-type allele, and the lower band (2.0 kb) to the mutant allele.

FIG. 1D: IRAK-M deficiency in homozygous mice. Total mRNA of macrophages were prepared from wild-type and homozygous animals and expression of irak-M mRNA was examined using Northern blotting and the irak-M specific ³²P-labeled probe.

FIGS. 2A-2C. Increased Cytokine Production of IRAK-M deficient Macrophages upon PAMP Stimulation.

FIG. 2A: Increased production of IL-12 p40 by IRAK-m deficient macrophages upon PAMP stimulation. Bone marrow derived macrophage were prepared from wild-type (white bar) and IRAK-M deficient mice (black bar) and plated in 24 well plates at the density of 2×10⁵ cells/well. Cells were stimulated with 10 μM of CpG oligo DNA (CpG), 10 μg/ml of mannan (MAN), 10 μg/ml of zymosan (ZYM), 10 μg/ml of double-stranded RNA (poly(IC)), 10 μg/ml of peptidoglycan (PGN), 1 of 10 ng/ml of LPS. 10 μg/ml of lipid A, 1 or 10 μg/ml of lipoteichoic acid (LTA), or medium alone (MED). 24 hours after stimulation, the concentration of IL-12 p40 in the supernatant was examined by ELISA. Experiments were repeated at least three times in triplicate with similar results. N.D.: not detected.

FIG. 2B: Increased production of TNFα by IRAk-M deficient macrophages upon PAMP stimulation. Bone marrow derived macrophages were prepared from wild-type (white bar) and IRAK-M deficient mice (black bar) and stimulated as in (A). 24 hours after stimulation, the concentration of TNFα in the supernatant was examined by ELISA. Experiments were repeated at least three times in triplicate with similar results. N.D.: not detected.

FIG. 2C: Increased production of IL-6 by IRAK-M deficient macrophages upon PAMP stimulation. Bone marrow derived macrophage were prepared from wild-type (white bar) and IRAK-M deficient mice (black bar) and stimulated as in (A). 24 hours after stimulation, the concentration of IL-6 in the supernatant was examined by ELISA. Experiments were repeated at least three times in triplicate with similar results. N.D.: not detected.

FIGS. 3A-3E. Increased Response of IRAK-M deficient Mice upon Bacterial Challenge in vitro.

FIG. 3A: Increased production of IL-12 p40 by IRAK-M deficient macrophages upon gram negative bacterial challenge. Bone marrow derived macrophages were prepared from wild-type and IRAK-M deficient mice. Cells were infected with Salmonella typhimurium (strain: S161 and S1230) or Echerichia coli (strain: DH5α as described in the Examples. HK: heat-killed bacteria. 24 hours after infection, the concentration of IL-12 p40 in the supernatant was examined by ELISA. N.D.: not detected.

FIG. 3B: Increased production of IL-6 by IRAK-M deficient macrophages upon gram negative bacterial challenge. Wild-type and IRAK-M deficient macrophages were prepared and infected with with Salmonella typhimurium (S161 and S1230) or Echerichia coli (DH5α) as described in (A). 24 hours after infection, the concentration of IL-6 in the supernatant was examined by ELISA. N.D.: not detected.

FIG. 3C: Increased production of TNFα by IRAK-M deficient macrophages upon gram negative bacterial challenge. Wild-type and IRAK-M deficient macrophages were prepared and infected with with Salmonella typhimurium (S161 and S1230) or Echerichia coli (DH5α) as described in (A). 24 hours after infection, the concentration of TNFα in the supernatant was examined by ELISA. N.D.: not detected.

FIG. 3D: Increased production of IL-12 p40 by IRAK-M deficient macrophages upon gram positive bacterial challenge. Bone marrow derived macrophages were prepared from wild-type and IRAK-M deficient mice. Cells were infected with Listeria monocytogenes as described in the Examples. HK: heat-killed bacteria. 24 hours after infection, the concentration of IL-12 p40 in the supernatant was examined by ELISA. N.D.: not detected.

FIG. 3E: Increased production of IL-6 by IRAK-M deficient macrophages upon gram positive bacterial challenge. Wild-type and IRAK-M deficient macrophages were prepared and infected with Listeria monocytogenes as described in (D). 24 hours after infection, the concentration of IL-6 in the supernatant was examined by ELISA. N.D.: not detected.

FIGS. 4A-4C: IRAK-M is induced by endotoxin and is required for endotoxin tolerance

FIG. 4A: Induction of irak-M mRNA by LPS stimulation in macrophages. Bone marrow derived macrophages were prepared and stimulated with 10 ng/ml of LPS for indicated periods. Total RNA samples were prepared and the expression of mRNA of irak-M, irak and HPRT were examined by Northern blotting analysis using irak, irak-M and HPRT specific ³²P labeled DNA probes. Hypoxanthine phosphoribosyltransferase (HRPT) was used as an internal control.

FIG. 4B: Induction of the expression of IRAK-M protein by LPS stimulation in macrophages. Bone marrow derived macrophages were prepared and stimulated with 10 ng/ml of LPS for indicated periods. Cell lysates were prepared and the expression of IRAK-M, IRAK, MyD88 and TRAF6 were examined by Western blotting analysis using anti-IRAK-M, anti-IRAK, anti-MyD88 and anti-TRAF6 antibodies.

FIG. 4C: Perturbed endotoxin tolerance in IRAK-M deficient macrophages. Bone marrow derived macrophages were prepared from wild-type or IRAK-M deficient mice. Endotoxin tolerance was induced by preactivation with 10 or 100 ng/ml of LPS (1^{st} LPS). After the indicated incubation period, cells were washed and stimulated again with 10 ng/ml of LPS (2^{nd} LPS). 24 hours after 2^{nd} stimulation of LPS, the concentration of IL-6 , IL-12 p40 and TNFα in the supernatant was examined by ELISA. The concentration of cytokines in each sample was compared to the sample with 2^{nd} stimulation alone and percentages of the cytokine production were presented. White bar: wild-type macrophages. Black bar: IRAK-M deficient macrophages.

FIGS. 5A-5B. Model for the regulation of TLR signaling by IRAK-M

FIG. 5A: Activation of IRAK upon TLR stimulation in the absence of IRAK-M. PAMPs stimulation of TLR may induce multimerization of these receptors which in turn causes recruitment of MyD88 and IRAK to TLRs (1). Proximity of IRAK or other kinases cause auto-or cross-phosphorylation (2). The phosphorylation of IRAK causes its conformational change (3). The conformational change of IRAK results in reduced affinity for the TLR signaling complex and IRAK is released to activate downstream molecules. Other adapter molecules in TLRs, Tollip (Burns et al., 2000) and Tirap/Mal (Fitzgerald et al., 2001; Horng et al., 2001) were abbreviated from the figure for readability.

FIG. 5B: Inhibition of TLR signaling by IRAK-M. In the presence of IRAK-M, TLR stimulation by PAMPs results in the recruitment of not only IRAK but also IRAK-M to the signaling complex which inhibits release of IRAK from the TLR signaling complex by either inhibition of phosphorylation of IRAK or stabilizing the TLR/MyD88/IRAK complex and therefore blocks downstream signaling.

### DETAILED DESCRIPTION OF THE INVENTION

The present invention relates to isolated nucleic acid encoding a murine IRAK-M protein, such as nucleic acid comprising the nucleic acid sequence depicted in SEQ ID NO.: 1 and isolated nucleic acid that encodes a murine IRAK-M protein comprising the amino acid sequence depicted in SEQ ID NO.: 2. It further relates to isolated IRAK-M protein encoded by the nucleic acid sequence depicted in SEQ ID NO.: 1 and isolated IRAK-M protein comprising the amino acid sequence depicted in SEQ ID NO.: 2. In further embodiments, the invention is an expression vector comprising the nucleic acid which has the sequence of SEQ ID NO.: 1 or an expression vector comprising nucleic acid encoding the amino acid sequence of SEQ ID NO.: 2. The expression vectors can further comprise DNA sufficient for expression of the DNA encoding the amino acid sequence depicted in SEQ ID NO.: 2 in cells. Also the subject of the invention are cells transformed with the vectors; isolated cells (e.g., mouse, human, other mammalian) that do not comprise nucleic acid encodive functional IRAK-M and isolated IRAK-M⁻¹⁻ cells. Such cells can be, for example, macrophages (mouse, human, other mammalian). They can comprise exogenous nucleic acid encoding IRAK-M (introduced into the cells or ancestors thereof) that is expressed. IRAK-M⁻¹⁻ cells of the present invention can be obtained from an IRAK-M deficient (IRAK-M⁻¹⁻) transgenic nonhuman animal (e.g., a mouse).

The invention is also a method for producing murine IRAK-M, comprising culturing cells that contain a vector comprising DNA encoding murine IRAK-M under conditions appropriate for expression of the DNA, wherein murine IRAK-M is thereby produced.

The invention is also a method of identifying a compound that modulates the innate immune response in an individual, comprising combining cells expressing murine IRAK-M with a candidate compound, and determining whether the candidate compound modulates IRAK-M activity in the cells, wherein modulation of IRAK-M activity in the cells by the candidate compound indicates that the candidate compound modulates the innate immune response in the individual. In one embodiment, the cells and candidate compound are combined under conditions appropriate for entry of the candidate compound into the cells.

The method can further comprise comparing IRAK-M activity in the presence of the candidate compound with IRAK-M activity for a standard deficient in IRAK-M activity, wherein IRAK-M activity in the presence of the candidate compound which is comparable to IRAK-M activity for the standard indicates that the candidate compound is an IRAK-M inhibitor. The method can be carried out in cells do not express IRAK-M.

In one embodiment, inhibition of IRAK-M activity in the cells by the candidate compound indicates that the compound inhibits IRAK-M activity and a compound that enhances the innate immune response is identified. The innate immune response identified can be, for example, production of inflammatory cytokines or chemokines.

The present invention also encompasses a method of identifying a compound that produces an immunoinhibitory effect in a subject, comprising combining cells expressing IRAK-M with a candidate compound and determining whether the candidate compound enhances IRAK-M activity in the cells. If enhancement of IRAK-M activity occurs in the cells, a candidate compound that produces an anti-inflammatory effect and an immunoinhibitory effect is identified.

In a further embodiment, the invention is a method of identifying a compound that produces an immunostimulatory effect in a subject, comprising combining cells expressing IRAK-M with a candidate compound and determining whether the candidate compound inhibits IRAK-M activity in the cells. If inhibition of IRAK-M activity occurs in the cells, a compound that produces an immunostimulatory effect is identified.

In another embodiment, the invention is a method of producing an anti-inflammatory effect and an immunoinhibitory effect in an individual, comprising administering to the individual a compound that enhances IRAK-M in cells in sufficient quantity to enhance IRAK-M, thereby producing an anti-inflammatory effect and an immunoinhibitory effect in the individual.

The invention further relates to a method of treating an inflammatory condition in an individual, comprising administering to the individual a compound that enhances IRAK-M activity in the cells in the individual, thereby producing an anti-inflammatory effect in the individual. The inflammatory condition can be, for example, an autoimmune condition, such as rheumatoid arthritis or lupus erythematosis.

The invention also relates to a method of determining whether a compound is an IRAK-M inhibitor. The method comprises: (a) contacting a cell expressing IRAK-M with a candidate compound and measuring the production by the cell of an inflammatory cytokine or chemokine upon stimulation with a TLR or IL-1R ligand; (b) comparing production by the cell of the inflammatory cytokine or chemokine in (a) with production by the cell of the inflammatory cytokine or chemokine in the absence of the candidate compound; (c) contacting a cell which does not express IRAK-M with the candidate compound and measuring production by the cell of an inflammatory cytokine or chemokine upon stimulation with a TLR or IL-1R ligand; and (d) comparing production by the cell of the inflammatory cytokine or chemokine in (c) with production by the cell of the inflammatory cytokine or chemokine in the absence of the candidate compound. If production in (a) is more than production in (b), and the production in (c) is comparable to production in (d), the compound is an IRAK-M inhibitor. In one embodiment of the method, the TLR or IL-1R ligand is capable of increasing production of an inflammatory cytokine.

In a further aspect, the invention is a method of determining whether a compound is an IRAK-M inhibitor comprising: (a) contacting a cell expressing IRAK-M with the candidate compound and measuring production by the cell of an inflammatory cytokine or chemokine upon stimulation with a pathogen (e.g., Salmonella typhimurium, Escherichia coli or Listeria monocytogenes); (b) comparing production by the cell of the inflammatory cytokine or chemokine of step (a) with production by the cell of the inflammatory cytokine (e.g., IL-1β, IL-6 , TNFα or IL-12) or chemokine in the absence of the candidate compound; (c) contacting a cell which does not express IRAK-M with the candidate compound and measuring production by the cell of an inflammatory cytokine or chemokine upon stimulation with a pathogen; (d) comparing production by the cell of the inflammatory cytokine or chemokine in step (c) with production by the cell of the inflammatory cytokine or chemokine in the absence of the candidate compound. If production in (a) which is more than production in (b), and production in (c) which is comparable to the production in (d) indicates that the compound is an IRAK-M inhibitor.

The invention is also a method of determining whether a compound is an IRAK-M inhibitor comprising: (a) contacting a cell expressing IRAK-M with the candidate compound and measuring NF-κB activation in the cell; (b) comparing NF-κB activation measured in step (a) with activation of NF-κB measured in a cell expressing IRAK-M in the absence of the candidate compound; (c) contacting a cell which does not express IRAK-M with the candidate compound and measuring activation of NF-κB in the cell; and (d) comparing NF-κB activation measured in step (c) with NF-κB activation measured in a cell which does not express IRAK-M in the absence of the candidate compound, wherein activation measured in (a) is more than the activation measured in (b), and activation measured in (c) is comparable to the activation measured in (d) indicates that the compound is an IRAK-M inhibitor. NF-κB activation (which can be increased upon TCR stimulation) is determined, for example, by examining the phosphorylation state of p38, IκBα, ERK1/2 or JNK or is detected by measuring IκBα degradation.

A further embodiment is a method of detecting an agonist of IRAK-M activity, comprising: (a) contacting a cell expressing IRAK-M with a candidate compound and measuring production of an inflammatory cytokine or chemokine upon stimulation with a TLR or IL-1R ligand; and (b) comparing production by the cell of an inflammatory cytokine or chemokine in (a) with the production by the cell of the inflammatory cytokine or chemokine in the absence of the candidate compound, wherein production in (a) which is less than production in (b) indicates that the compound is an IRAK-M agonist.

The invention is also a method of detecting or identifying an agonist of IRAK-M activity, comprising: (a) contacting a cell expressing IRAK-M with a candidate compound and measuring production of an inflammatory cytokine or chemokine upon stimulation with a pathogen; and (b) comparing production by the cell of an inflammatory cytokine or chemokine in (a) with the production by the cell of the inflammatory cytokine or chemokine in the absence of the candidate compound, wherein production in (a) which is less than production in (b) indicates that the compound is an IRAK-M agonist and an agonist of IRAK-M activity is identified.

The invention further relates to a method of determining whether a compound is an IRAK-M agonist comprising: (a) contacting a cell expressing IRAK-M with the candidate compound and measuring the NF-κB activation in the cell; (b) comparing the NF-κB activation measured in step (a) with the activation of NF-κB measured in a cell expressing IRAK-M in the absence of the candidate compound, wherein activation measured in (a) which is less than activation measured in (b) indicates that the compound is an IRAK-M agonist.

The innate immune system is a host defense mechanism which is conserved evolutionarily from plants to humans (Medzhitov and Janeway, 1997). Essential components of the innate immune system are Toll-like receptors (TLRs) which recognize various microbial products termed PAMPs (pathogen associated molecular pattern). Recognition of these PAMPs leads to the activation of the innate immune system which in turn activates adaptive immunity (Medzhitov and Janeway, 1997). Recent findings revealed that TLRs recognize specific PAMPs through their extracellular domains termed LRR (leucine rich repeat); TLR2, TLR3, TLR4, TLR5, TLR6 and TLR9 recognize the gram-positive bacterial products peptidoglycan, double-stranded RNA, the gram-negative bacterial product LPS, the flagellar components Flagellin, mycoplasmal macrophage-activating lipopeptide-2 kD (MALP-2) and CpG bacterial DNA respectively (Alexopoulou et al., 2001; Hayashi et al., 2001; Hemmi et al., 2000; Hoshino et al., 1999; Poltorak et al., 1998; Qureshi et al., 1999; Takeuchi et al., 1999). Several different components are involved in TLR signaling. The adapter molecule, termed MyD88 has dual binding domains, a TIR domain (Toll and IL-1 Receptor homology domain) and a death domain (DD), and binds to the intracellular TIR domain of TLRs (Medzhitov et al., 1998; Wesche et al., 1997). Upon TLR stimulation, a death domain carrying serine/threonine kinase IRAK is recruited to the TLR signaling complex via the DD-DD interaction (Medzhitov et al., 1998). IRAK is phosphorylated either by autophosphorylation or cross phosphorylation (Cao et al., 1996; Wesche et al., 1999), losing affinity for the TLR signaling complex. Consequently, IRAK is released from the complex permitting binding to downstream molecules such as TRAF6, resulting in the activation of NF-κB, JNK, p-38 and ERK1/2 (Kawai et al., 1999; Medzhitov et al., 1998; Wesche et al., 1997; Zhang et al., 1999). The finding of two other IRAK family proteins, IRAK-2 and IRAK-M, has added complexity to this signaling model (Muzio et al., 1997; Wesche et al., 1999). Similar to IRAK, IRAK-2 is expressed ubiquitously (Muzio et al., 1997). However, the expression of IRAK-M is restricted to monocytes/macrophages and is found in only low amounts in other tissues; it was therefore termed IRAK-M (Wesche et al., 1999). IRAK-2 and IRAK-M have no active kinase activity but they can still activate NF-κB by overexpression in 293T cells and restore IL-1 signaling in IRAK-deficient cells by transfection, with a reduced efficiency compared to wild-type IRAK (Muzio et al., 1997; Wesche et al., 1999). Although it has been shown that MyD88-deficient cells are totally incompetent to produce cytokines upon TLR stimulation (Kawai et al., 1999), null mutation of IRAK by gene-targeting resulted in the partial reduction of cytokine production by LPS stimulation (Swantek et al., 2000), suggesting that IRAK-2 or IRAK-M may play a redundant role in TLR signaling.

Although the inflammatory response is critical to control the growth of pathogenic microorganisms(Cross et al., 1995; Eden et al., 1988; Hagberg et al., 1984; Shahin et al., 1987), excessive production of proinflammatory cytokines is harmful to the host and in extreme cases can be fatal (Beutler et al., 1985; Danner et al., 1991). Animals or humans chronically (or repeatedly) exposed to endotoxin (or LPS) such as in patients with bacteremia exhibit a transient increase in the threshold to endotoxin challenge (Beeson, 1947; Greisman et al., 1966; Ziegler-Heitbrock, 1995). This phenomenon is called endotoxin tolerance and it is regarded as a defense mechanism to protect the host organism from endotoxin shock (Gustafson et al., 1995; Henricson et al., 1990; Salkowski et al., 1998). Endotoxin tolerance provides an important negative feedback mechanism from inflammatory response which regulates the sensitivity of immune system to pathogens or PAMPs. Recent findings revealed that several factors are involved in this mechanism such as the down-regulation of TLR4 (Nomura et al., 2000) and decreased activation of NF-κB (Goldring et al., 1998; Kastenbauer and Ziegler-Heitbrock, 1999; Ziegler-Heitbrock et al., 1994). However, the mechanism underlying this phenomenon is largely unknown. To investigate the role of IRAK-M in TLR signaling, we generated IRAK-M deficient mice using gene targeting in mouse embryonic stem (ES) cells. The expected phenotype of IRAK-M deficiency was a reduction of the innate immune response. Surprisingly, however, the innate response was strongly enhanced in IRAK-M deficient mice showing that IRAK-M negatively regulates TLR signaling. Furthermore IRAK-M deficient cells have strikingly impaired endotoxin tolerance, indicating that IRAK-M is essential to control the innate immune system via this negative feedback mechanism.

The present invention is illustrated by the following examples, which are not intended to be limiting in any way.

### EXAMPLE 1

### Molecular Cloning and Generation of IRAK-M Deficient Mice

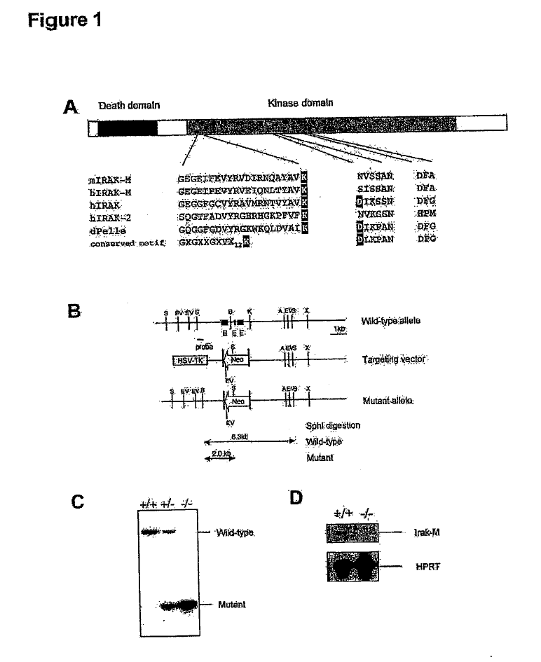

A homology search for IRAK homologues in the EST data bases and extension of the coding sequence by 5′-RACE resulted in the molecular cloning of the full length cDNA encoding a novel mouse kinase of 596 amino acids and a calculated molecular mass of 68.7 kDa. BLAST search revealed that this kinase is the murine orthologue of human IRAK-M sharing 73% identities in its amino acid sequence. Mouse IRAK-M has 12 serine/threonine kinase subdomains and a conserved lysine in the ATP binding site in subdomain II; but mouse IRAK-M lacks the catalytically active aspartate in subdomain VIB as does human IRAK-M (FIG. 1A), suggesting that mouse IRAK-M does not have active kinase activity. To assess the physiological role of IRAK-M in TLR signaling, we generated IRAK-M-deficient mice by homologous recombination in embryonic stem (ES) cells. A gene-targeting construct was generated to replace two thirds of the kinase domain with a neomycin-resistance gene (neo) (FIG. 1B). Homologous recombination in ES cells was confirmed by Southern blot analysis (FIG. 1C), and the absence of IRAK-M expression in homozygous animals was confirmed by Northern blot (FIG. 1D). IRAK-M-deficient mice were born at the expected mendelian ratio and showed no gross developmental abnormalities and a normal complement of lymphocytes as determined by flow cytometry (data not shown).

### EXAMPLE 2

### Enhanced Response in IRAK-M Deficient Macrophages upon TLR Stimulation

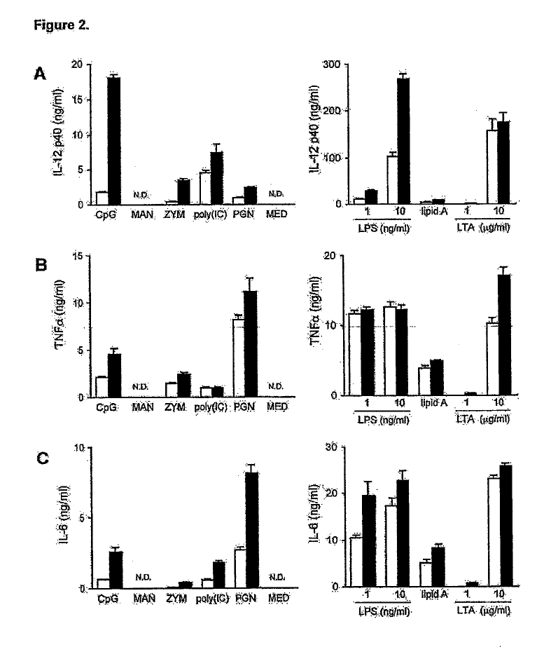

To characterize the effect of IRAK-M deficiency in TLR signaling, IRAK-M deficient macrophages were prepared from bone marrow and stimulated with various PAMPs for 6 and 24 hours. Contrary to our expectations, IRAK-M deficient macrophages revealed significantly increased production of IL-12 p40, IL-6 and TNFα when compared to wild-type macrophages at both time points, 24 hours (FIGS. 2A, B and C) and 6 hours after stimulation (data not shown). Interestingly, although IRAK-M deficiency affected signaling by all TLRs tested, it had the strongest effect on TLR9, which is a receptor for CpG DNA.

### EXAMPLE 3

### Increased Inflammatory Responses of IRAK-M Deficient Mice Challenged with Bacteria in Vitro and in Vivo

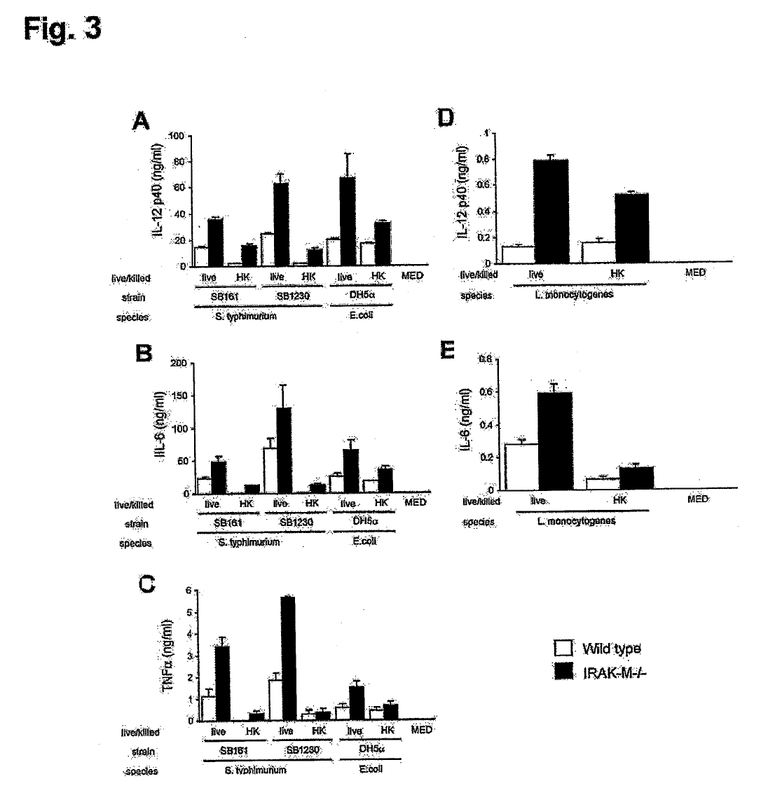

In order to investigate the physiological roles of IRAK-M in host defense, we infected IRAK-M deficient macrophages with gram-negative and gram-positive bacteria. IRAK-M macrophages were infected with two gram negative bacteria, Salmonella typhimurium and Escherichia coli, and cytokine production was assessed in the cell supernatants at 6 and 24 hours after infection using ELISA. Because wild-type S. typhimurium rapidly kills macrophages via their type III secretion system (Chen et al., 1996b), we used two mutant strains, SB161 and SB1230 whose type III secretion system was mutated. IRAK-M deficient macrophages challenged with live or heat killed gram-negative bacteria, S. typhimurium and E. coli, produced significantly increased amounts of IL-12p40, IL-6 and TNFα at 24 hours (FIGS. 3ABC) and 6 hours (data not shown) after infection, compared to control cell. IRAK-M macrophages were also challenged with the gram-positive bacterium, Listeria monocytogenes and cytokine production was analyzed at 6 and 24 hours after infection. IRAK-M deficient macrophages produced increased levels of the cytokines, IL-12 p40 and IL-6, upon treatment with either live or heat-killed L. monocytogenes at 24 hours (FIGS. 3DE) and 6 hours after infection.

To investigate the role of IRAK-M in host defense against bacterial infection, Applicants infected IRAK-M deficient mice with a virulent strain of S. typhimurium. Applicants chose a strain (SB161) of S. typhimurium that although virulent in a mouse model of infection, is significantly reduced in its ability to cause intestinal pathology by virtue of carrying a mutation that renders it deficient in type III secretion (Galan and Curtiss, 1989; Penheiter et al., 1997). IRAK-M deficient mice were infected with S. typhimurium orally and sacrificed 72 hours later to assess the intestinal inflammation and bacterial numbers in spleen. IRAK-M deficient mice challenged with S. typhimurium showed grossly enlarged large Peyer's patches. Furthermore, the actual number of enlarged Peyer's patches was significantly increased in IRAK-M deficient mice compared to the wild-type. Histological examination of Peyer's patches in IRAK-M deficient mice infected with S. typhimurium revealed severe inflammatory infiltrates in Peyer's patches with numerous polymorphonuclear cells and accompanying hemorrhage, in significant contrast to wild-type mice which showed only mild inflammation of their Peyer's patches. The bacterial organ load was examined using spleens of infected mice. In spite of the increased inflammatory response in the gut, the number of bacterial colony forming units (CFU) in spleens of infected IRAK-M deficient mice were not increased compared to the wild-type mice, suggesting that the increased inflammatory response in IRAK-M deficient mice was due to enhanced innate immunity itself rather than the enhanced susceptibility to bacterial infection.

### EXAMPLE 4

### Enhanced TLR Signaling by IRAK-M Deficiency

TLR stimulation activates NF-κB, JNK, p38 and ERK1/2 through the signaling molecules MyD88 and IRAK (Kawai et al., 1999; Medzhitov et al., 1998). Applicants therefore examined the activation of these downstream effectors of TLR signaling in IRAK-M deficient cells. IRAK-M deficient macrophages were stimulated with CpG DNA or LPS for 10, 20 and 60 minutes and the activation of NF-κB, JNK, p38 and ERK1/2 was analyzed by examining their phosphorylation state with specific antibodies. CpG stimulation of IRAK-M deficient macrophages showed rapid phosphorylation and degradation of IκBα compared to wild-type cells. Phosphorylation of JNK, p-38 and ERK1/2 in IRAK-M deficient macrophages showed faster and stronger activation than that of wild-type cells, indicating enhanced signaling in CpG stimulated IRAK-M deficient macrophages and suggesting that IRAK-M negatively regulates these signaling pathways. LPS stimulated IRAK-M deficient macrophages also showed enhanced signaling to NF-κB, JNK, p-38 and ERK, although the augmentation was not as great as that seen in CpG stimulated cells. Bone marrow-delivered macrophages were stimulated 10 ng/ml of TNFα for 0, 5, 10 and 30 minutes. Cell lysates were blotted with anti-phospho-IkBα, anti-phospho-JNK, anti-JNK, anti-phospho-p38, anti-phospho-ERK1/2, and anti-ERK1/2 antibodies. No enhancement of signaling in IRAK-M -/-macrophages was observed upon TNFα stimulation.

### EXAMPLE 5

### IRAK-M is Required for Endotoxin Tolerance.

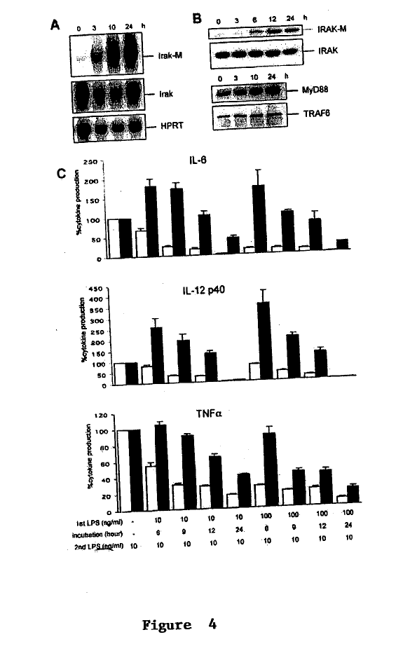

Applicants results showing that IRAK-M is a negative regulator of TLR signaling led them to consider the possibility that IRAK-M might be involved in the induction of endotoxin tolerance. If this were the case, IRAK-M would be expected to be initially present at low levels, but then to be increased in amount following stimulation with PAMPs. To examine this possibility, wild-type macrophages were stimulated with LPS, and the levels of irak-M and irak mRNA were assessed by Northern blotting. As shown in FIG. 4A, irak-M mRNA was significantly induced by LPS stimulation whereas irak mRNA was not induced. The protein levels of IRAK-M, IRAK, MyD88 and TRAF6 were also examined by Western blotting. Consistent with the result of Northern blotting analysis, the expression of IRAK-M was induced by LPS whereas the expression of IRAK, MyD88 and TRAF6 were not (FIG. 4B). We next determined the ability of IRAK-M deficient macrophages to develop endotoxin tolerance. IRAK-M deficient macrophages were first stimulated with 10 or 100 ng/ml of LPS (primary LPS stimulation). After incubation for the indicated periods, cells were re-stimulated with 10 ng/ml of LPS (second LPS stimulation) and cytokine production was examined by ELISA at 24 hours after secondary LPS stimulation. Cytokine levels at each time point were compared to the cytokine level of macrophages which received only the second LPS stimulation. As shown by previous studies (Nomura et al., 2000), wild type macrophages showed reduced cytokine production in accordance with a longer incubation time and a higher dose of LPS (FIG. 4C), indicating that endotoxin tolerance is dependent on the incubation time and dose of the primary LPS treatment. IRAK-M deficient macrophages, however, showed a lack of endotoxin tolerance and consequently the levels of cytokine produced upon LPS re-stimulation were not decreased as much as in re-stimulated wild-type macrophages (FIG. 4C). IL-6 and TNFα production after short incubation times (6 and 9 hours) was even increased compared to that of non-pretreated macrophages, indicating that IRAK-M is essential for endotoxin tolerance and that the absence of this negative regulator causes abnormal enhancement of inflammatory cytokine production. After 24 hours of incubation, however, IRAK-M deficient macrophages showed reduced IL-6 and TNFα production and almost no IL-12p40 production, suggesting that there is a possible second mechanism to mediate endotoxin tolerance which still operates at later time points in IRAK-M deficient cells.

### EXAMPLE 6

### Inhibition of TLR Signaling by IRAK-M

Data presented herein show that IRAK and IRAK-M play completely different roles in TLR signaling. IRAK is a positive signal transducer whereas IRAK-M is a negative regulator. Although both molecules share a similar structure, IRAK-M lacks kinase activity (Cao et al., 1996; Wesche et al., 1999). Applicants therefore hypothesized that the difference in the functions of these two signaling molecules may at least be due in part to the difference in their kinase activities. To test this, various IRAK family proteins were transduced into IRAK-M deficient macrophages using a retroviral vector carrying an IRES-GFP expression cassette. GFP positive cells were sorted and stimulated with LPS. IRAK-M transduced macrophages produced significantly reduced levels of TNFα, suggesting that IRAK-M overexpression inhibits cytokine production, which is consistent with its negative regulatory role. Transduction of kinase activity dead IRAK (IRAKKD, K206/A mutation) and IRAK2 also resulted in reduced TNFα production, but transduction of wild-type IRAK did not reduce the TNFα production level.

Next, Applicants tested whether IRAK-M could inhibit recruitment of IRAK to the TLR signaling complex by virtue of potential dominant negative effects. Applicants cotransfected HA-tagged MyD88, Flag-tagged IRAKKD and Flag-tagged IRAK-M into 293T cells. After immunoprecipitation using anti-HA antibody, MyD88 associated molecules were analyzed by Western blotting and anti-Flag antibody. Cotransfection of MyD88 and IRAK resulted in the association of these two molecules. Cotransfection of IRAK-M together with MyD88 and IRAK resulted in enhanced, rather than decreased association of IRAK with MyD88, suggesting that IRAK-M does not inhibit the recruitment of IRAK to MyD88. Furthermore, Applicants tested whether phosphorylated IRAK associates with MyD88. Wild-type IRAK and MyD88 were cotransfected and their association was examined. Overexpression of wild-type IRAK causes the appearance of slowly migrating bands which reflects IRAK autophosphorylation (Cao et al., 1996; Wesche et al., 1999; Yamin and Miller, 1997). Transfection of wild-type IRAK and MyD88 resulted in readily detectable slowly migrating bands and only a low level of the faster migrating band, suggesting that most IRAK was phosphorylated under these conditions. Co-immunoprecipitation studies showed that phosphorylated IRAK did not associate with MyD88 In contrast, cotransfection of IRAK-M together with MyD88 and wild-type IRAK resulted in increased relative levels of the faster migrating (unphosphorylated) band, suggesting that IRAK-M may inhibit phosphorylation of IRAK. These immunoprecipitation studies also detected an enhanced association of IRAK and MyD88. Notably, even phosphorylated IRAK, which has little binding affinity for MyD88, also remained associated with MyD88 in the presence of IRAK-M, suggesting that IRAK-M may increase the affinity of both phosphorylated and unphosphorylated forms of IRAK for MyD88.

Discussion

Innate immunity is the first line of host defense against pathogenic microorganisms (Medzhitov and Janeway, 1997). The TLR system has been recently highlighted as an essential detector of pathogens or PAMPs. The innate immune system stimulated via TLR activates the adaptive immune system by the production of proinflammatory cytokines such as IL-1β, IL-6, TNFα or IL-12 and the induction of key surface molecules, which drive T cell activation including MHC, CD40, CD80 or CD86 (Akira et al., 2001; Medzhitov and Janeway, 1997; Schnare et al., 2001). Cytokine production, however, has a pronounced positive feedback mechanism in the immune system which, if left unchecked, can cause severe immunopathology. Indeed a number of pathologies such as Crohn's and inflammatory bowel disease have been postulated to be the result of disregulated innate immune responses (Van Heel et al., 2001). However, the actual mechanisms by which the innate immune system is held in check to prevent immunopathology are largely unknown.

Applicants have shown here that the kinase IRAK-M exerts a critical negative regulatory role in the innate immune system. Consistent with this negative regulatory function, macrophages from IRAK-M deficient mice exhibited an enhanced production of pro-inflammatory cytokines when infected with either live or dead bacteria (FIGS. 3A-E). Furthermore, IRAK-M deficient mice showed a greatly exacerbated intestinal inflammatory response to challenge with the enteric pathogenic bacteria Salmonella typhimurium. In comparison to wild type, infected IRAK-M deficient mice exhibited severely enlarged and inflamed Peyer's patches, which is the site of Salmonella colonization of the intestinal track. The exacerbated response of IRAK-M deficient mice is likely the result of enhanced TLR signaling. Consistent with this hypothesis, IRAK-M deficient macrophages stimulated with known agonists of TLRs such as LPS or CpG DNA displayed increased NF-κB and MAP kinase activation, which are well-characterized outputs of TLR stimulation (Kawai et al., 1999; Medzhitov et al., 1998; Zhang et al., 1999).

Persistent stimulation with LPS results in a phenomenom known as endotoxin tolerance whereby responses to this TLR agonist are dampened by poorly understood negative regulatory mechanisms. Results presented herein indicate that IRAK-M is a key component of this important control system. Consistent with this hypothesis, IRAK-M-deficient macrophages were significantly impaired in the development of tolerance upon repeated stimulation with LPS (FIG. 4C). Notably, however, IRAK-M macrophages retained some capacity to develop LPS tolerance suggesting the existence of additional regulatory mechanisms to control the response to LPS. The recently reported downregulation of TLR4 in peritoneal macrophages may be one such alternative mechanism (Nomura et al., 2000).

What is the mechanism by which IRAK-M exerts its function? A notable feature of IRAK-M is that despite its high degree of amino acid sequence similarity to IRAK, it lacks kinase activity (FIG. 1A and (Wesche et al., 1999)) and has a weak capacity to be phosphorylated (Wesche et al., 1999). It is therefore likely that these features are important for its negative regulatory role. However, the role of the kinase activity of IRAK in TLR signaling is the subject of some controversy. Indeed, kinase-inactive mutants of IRAK, as well as the kinase inactive forms IRAK-M and IRAK-2, can still activate NF-κB when overexpressed in cultured cells (Knop and Martin, 1999; Maschera et al., 1999; Muzio et al., 1997; Wesche et al., 1999). Furthermore, kinase-deficient IRAK mutant can restore NF-κB activation in IRAK deficient cells upon stimulation with IL-1β (Knop and Martin, 1999; Li et al., 1999). Applicants' studies showed that in contrast to IRAK-M and IRAK-2 or IRAKKD, expression of the wild-type-kinase-active IRAK failed to suppress cytokine production upon LPS stimulation. These results indicate that the autophosphorylation is important for signaling by this kinase family.

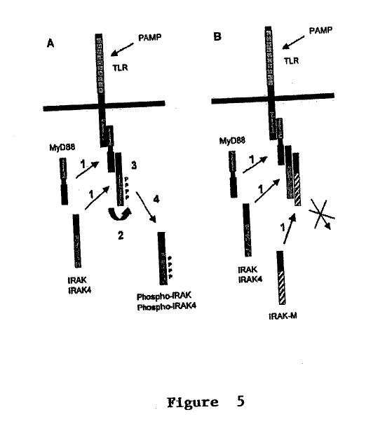

Applicants propose the following model for IRAK-M function Activation of TLR by PAMPs may dimerize these receptors, following which IRAK and the adapter protein Myd88 are recruited to the receptors resulting in the activation of IRAK and its subsequent phosphorylation (FIG. 5A). IRAK phosphorylation results in a conformational change losing its affinity for the TLR signaling complex and thereby allowing the stimulation of downstream signaling pathways through its association with signaling molecules such as TRAF6. IRAK-M presumably inhibits this process by either inhibiting the phosphorylation of IRAK or its dissociation from the TLR signaling complex (FIG. 5B). Despite their lack of kinase activity, IRAK-M and IRAK-2 have been reported to be able to complement NF-κB activation in IRAK deficient cells to some degree, although much less effectively than wild-type IRAK (Wesche et al., 1999). In the context of this model we propose that this may occur upon their phosphorylation by another kinase(s) that may be present in the TLR signaling complex.

Like IRAK-M, IRAK-2 may also function as a negative regulator of TLR signaling. Indeed, these two proteins share many features; they lack kinase activity (FIG. 1A and (Muzio et al., 1997; Wesche et al., 1999)), there expression is induced by stimulation (FIG. 4A and (Wesche et al., 1999)), and they can reduce cytokine production upon LPS stimulation. However, these highly related proteins display a different pattern of tissue expression; while IRAK-M is preferentially expressed in monocytes/myeloid cells, IRAK2 is expressed ubiquitously (Muzio et al., 1997; Wesche et al., 1999). Because TLR expression is high in myeloid lineage cells and IL-1 receptors are expressed ubiquitously (McMahan et al., 1991; Muzio et al., 2000), it is conceivable that IRAK-M is the main regulator for TLR signaling whereas IRAK2 is a regulator for IL-1 signaling. Study using IRAK2 deficient mice should elucidate a role of IRAK2 in TLR/IL-1 signaling.

In summary, Applicants have identified IRAK-M as a negative regulator of TLR signaling. IRAK-M is required to induce endotoxin tolerance and the expression of IRAK-M is inducible by TLR stimulation, illustrating that IRAK-M is a key component of the feedback regulatory system of innate immunity. IRAK-M may therefore play a critical role in the maintenance of homeostasis of the innate immune system.

Experimental Procedures

The following procedures and materials were used in the work described herein.

Molecular Cloning and Expression Vectors

Full length mouse IRAK-M cDNA was obtained by 5′-RACE using an EST clone (accession number AA930623) using the primer 5′-cct ata tga gca acg gga cgc tt (SEQID No.: 3). Mammalian expression vectors encoding NH2-terminal Flag-tagged mouse IRAK and IRAKKD were a kind gift of Sankar Ghosh, Yale University. A construct encoding Flag-tagged human IRAK-M was a kind gift of Zaodan Cao, Tularik, Inc (Wesche et al., 1999). The retroviral expression vectors pCL-Eco and pCLXSN were purchased from Imgenex (La Jolla, Calif.). pCLXSN-IRESGFP was generated by inserting the Xba I-blunt/Xho I fragment from pSB965 (Chen et al., 1996a) into the BamH I-blunt/Xho I site of pCLXSN. The pCLXSN-IRESGFP encoding Flag-tagged IRAK-M, IRAK, IRAKKD or IRAK2 were constructed by insertion into EcoR I site of pCLXSN-IRESGFP with PCR products generated by 5′-cggaattcgccaccatggactacaaagacgatgacgacaagatggcggggaactgtggggcc (SEQID No.: 4)as a forward primer and 5′-ttattcttttttgtactgttcatattc (SEQID No.: 5) as a reverse primer (for IRAK-M), 5′accatggactacaaagacgatgacgacaagatggacgccctggagcccgccgac (SEQID No.: 6) as a forward primer and 5′-tcagctctgaaattcatcactttcttcagg (SEQID No.: 7) (for IRAK and IRAKKD) as a reverse primer, 5′-accatggactacaaagacgatgacgacaagatggcctgctacatctaccagctg (SEQID No.: 8) as a forward primer and 5′-ttatgtaacatcctggggaggctccagg (SEQID No.: 9) as a reverse primer (for IRAK2), respectively. Expression of Flag-tagged proteins was confirmed by Western blotting of transfected 293T cell lysates.

Generation of IRAK-M Deficient Mice

A 129SV/J genomic library (Stratagene) was screened with the murine irak-M cDNA to obtain a mouse irak-M genomic clone. Six phage carrying overlapping genomic clones encompassing irak-M were isolated. A targeting vector was designed to replace a 1.2 kb genomic fragment containing three exons encoding two third of the kinase domain with the loxP-flanked neomycin resistance (neo) gene expression cassette. The targeting vector was linearized with Not I and electroporated into W9.5 ES cells. Clones resistant to G418 and gancyclovir were selected, and homologous recombination was confirmed by Southern blotting. Eight out of 70 clones screened were positive for homologous recombination. Three clones homologous for the targeted mutation were injected into C57BL/6 blastocysts, which were subsequently transferred into pseudopregnant foster mothers. The resulting male chimeric mice were bred to C57BL/6 females to obtain heterozygous mice. Germline transmission of the mutant allele from all three original ES clones was verified by Southern blot analysis of tail DNA from F1 offspring with agouti coat color. Interbreeding of the obtained heterozygous mice was performed to generate homozygous IRAK-M deficient mice. Identical phenotype were obtained from all three lines.

Reagents

Lipopolysacchride (LPS) from Salmonella abortus equi, Lipid A from Escherichia coli, lipoteichoic acid (LTA) from Staphylococcus aureus, mannan from Saccharomyces cerevisiae and Zymosan A from Sacharomyces cerevisiae were purchased from Sigma. Peptidoglycan (PGN) from Staphylococcus aureus was from Fluka. Poly (I-C) double stranded RNA was from Amersham Pharmacia Biotech. Phosphorothioate-modified CpG oligo DNA (tccatgacgttcctgacgtt) was synthesized in the HHMI Biopolymer & W. M. Keck Biotechnology Resource Laboratory in Yale University. The anti-Flag M2 monoclonal antibody, anti-HA antibody and rabbit anti-IRAK-M antibody were purchased from Sigma, BabCO and Chemicon International respectively.

Culture of Bone Marrow Derived Macrophages

Bone marrow derived macrophages were prepared as described before (Celada et al., 1984). Briefly, bone marrow cells from tibia and femur were obtained by flushing with DMEM (Invitrogen). The complete medium was prepared with DMEM supplemented with 20% heat-inactivated fetal calf serum, glutamine (both from Invitrogen) and 30% L929 supernatant containing macrophage stimulating factor. Bone marrow cells were cultured in 10 ml of complete medium at an initial density of 4×10⁵ cells/ml in 100 mm Petri Dish (Becton Dickinson) at 37° C. in a humified 10% CO₂ atmosphere for 5 days. Five milliliters of the complete medium was added into the culture at day 3. Cells were harvested with cold DPBS (Invitrogen), washed, resuspended in DMEM supplemented with 10% of Fetal calf serum and used at a density of 2×10⁵/ml for experiments unless mentioned in the figure legends. Cells were left untreated for at least 4 h at 37° C. in 10% CO₂ prior to further handling.

Listeria Infection of Macrophages

The cells were cultured without antibiotics and listeria (ATCC strain 43251) were added at an MOI of 50 bacteria per macrophage. After incubation for 30 min, extracellular bacteria were removed by washing the cells three times with DPBS. To prevent reinfection, the cells were cultured in medium containing gentamicin sulfate (50 μg/ml, Invitrogen)

Salmonella and E. coli Infection of Macrophages in Vitro

The S. typhimurium strain SB161, which carries a nonpolar mutation in the invG gene, has been previously described (Kaniga et al., 1994). In vitro infection of macrophages with S. typhimurium has been described elsewhere (Chen et al., 1996b). Briefly macrophages were seeded without antibiotics in 24 well dishes at 2×10⁵ cells/well. Eighteen hours later macrophages were infected with SB161 or the E. coli strain DH5-α at an MOI of 50 bacteria per macrophage at 37° C. in DMEM+10% FBS. After 25 minutes macrophages were washed 3 times with HBSS and 100 ug/ml gentamicin was added to the media to kill any extracellular bacteria. Culture media was collected at 6 and 24 hours postinfection for cytokine measurements.

Salmonella Challenge of Mice in Vivo

Age and sex matched groups of mice were infected orally with Salmonella typhimurium strain SB161 at 109 bacteria per mouse. Mice were euthanized 72 hours after infection and analyzed. Enlarged Peyer's patches in small intestine were fixed with 10% formalin and stained by Hematoxilin and eosin (H&E). The spleen from each mouse was homogenized in 10 ml of BSG buffer, and serial dilutions of the homogenate were plated on LB/Strep agar plates. Plates were incubated at 37° C. for 18 hours and colony forming units (CFU) were counted.

Measurement of Cytokine Production from Macrophages

Bone marrow derived macrophages were cultured with indicated concentration of LPS, lipidA, LTA, PGN, mannan, Zymosan, poly(I-C), CpG DNA or media alone for 6 and 24 hours. In infection study, macrophages were infected with Salmonella typhimurium or Listeria monocytogenes, and cultured for 6 and 24 hours. The concentration of IL-12 p40, IL-6 and TNF-α in the culture supernatant was measured by ELISA.

Retoviral Infection of Macrophages

Replication-incompetent retroviral particles were generated using the RetroMax retroviral system (Imgenex, La Jolla, Calif.) Briefly 10 cm dishes of HEK293T cells were transfected by the calcium phosphate precipitation method with 10 ug of pCL-Eco and 10 ug of either pCLXSN-IRESGFP, pCLXSN-Flag-tagged IRAK-M -IRESGFP, pCLXSN-Flag-tagged IRAK-IRESGFP, pCLXSN-Flag-tagged IRAKKD-IRESGFP or pCLXSN-Flag-tagged IRAK2-IRESGFP. Viral supernatants were harvested 48 hours posttransfection and used to infect bone marrow derived macrophages at day 2 and day 3 of maturation. At day 5 GFP positive and negative cells were FACS sorted using a FACS Vantage machine (Becton Dickinson) and analyzed for cytokine production as described.

Northern Blot Analysis

Total RNA was isolated using TRIzol reagent (Invitrogen) according to the manufacturer's instruction. Total RNA (20 mg) was then separated by electrophoresis, blotted to a nitrocellulose membrane (Amersham) and probed with ³²P-labeled DNA probes. The irak, irak-M and HPRT specific probes were generated by PCR using forward primer 5′-gccagtggaaagtgatgagagtg (SEQID No.: 10) and reverse primer 5′-gaaaaagcctgatgacagcagttg (SEQID No.: 11) for murine irak, primers forward 5′-tccttcaggtgtccttctccactg (SEQID No.: 12) and reverse 5′-cctcttctccattggcttgctc (SEQID No.: 13) for murine irak-M, and primers forward 5′-gttggatacaggccagactttgttg (SEQID No.: 14) and reverse 5′-gagggtaggctggcctataggct (SEQID No.: 15) for HPRT.

Western Blot Analysis and Immunoprecipitation

Cell lysis, immunoprecipitation and blotting was carried as described before (Kobayashi et al., 1999). The membrane was blotted with an antibody to phosphorylated-IκBκ, IκBκ, phosphorylated-JNK, JNK, phosphorylated-p38, p38, phosphorylated-ERK1/2, ERK1/2 (Cell signaling), IRAK-1, TRAF6 (Santa Cruz), MyD88 (StressGen), IRAK-M (Chemicon International), FLAG-tag (Sigma) and HA-tag (BabCO).

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain, using no more than routine experimentation, many equivalents to the specific embodiments of the invention described specifically herein. Such equivalents are intended to be encompassed in the scope of the claims.

References

Akira, S., Takeda, K., and Kaisho, T. (2001). Toll-like receptors: critical proteins linking innate and acquired immunity, Nat Immunol 2, 675-80.

Alexopoulou, L., Holt, A. C., Medzhitov, R., and Flavell, R. A. (2001). Recognition of double-stranded RNA and activation of NF-kappaB by Toll-like receptor 3, Nature 413, 732-8.

Beeson, P. B. (1947). Tolerance to bacterial pyrogens. I. Factors influencing its development, J Exp Med 86, 29-38.

Beutler, B., Milsark, I. W., and Cerami, A. C. (1985). Passive immunization against cachectin/tumor necrosis factor protects mice from lethal effect of endotoxin, Science 229, 869-71.

Burns, K., Clatworthy, J., Martin, L., Martinon, F., Plumpton, C., Maschera, B., Lewis, A., Ray, K., Tschopp, J., and Volpe, F. (2000). Tollip, a new component of the IL-1RI pathway, links IRAK to the IL-1 receptor, Nat Cell Biol 2, 346-51.

Cao, Z., Henzel, W. J., and Gao, X. (1996). IRAK: a kinase associated with the interleukin-1 receptor, Science 271, 1128-31.

Celada, A., Gray, P. W., Rinderknecht, E., and Schreiber, R. D. (1984). Evidence for a gamma-interferon receptor that regulates macrophage tumoricidal activity, J Exp Med 160, 55-74.

Chen, L. M., Hobbie, S., and Galan, J. E. (1996a). Requirement of CDC42 for Salmonella-induced cytoskeletal and nuclear responses, Science 274, 2115-8.

Chen, L. M., Kaniga, K., and Galan, J. E. (1996b). Salmonella spp. are cytotoxic for cultured macrophages, Mol Microbiol 21, 1101-15.

Cross, A., Asher, L., Seguin, M., Yuan, L., Kelly, N., Hammack, C., Sadoff, J., and Gemski, P., Jr. (1995). The importance of a lipopolysaccharide-initiated, cytokine-mediated host defense mechanism in mice against extraintestinally invasive Escherichia coli, J Clin Invest 96, 676-86.

Danner, R. L., Elin, R. J., Hosseini, J. M., Wesley, R. A., Reilly, J. M., and Parillo, J. E. (1991). Endotoxemia in human septic shock, Chest 99, 169-75.

Eden, C. S., Shahin, R., and Briles, D. (1988). Host resistance to mucosal gram-negative infection. Susceptibility of lipopolysaccharide nonresponder mice, J Immunol 140, 3180-5.

Fitzgerald, K. A., Palsson-McDermott, E. M., Bowie, A. G., Jefferies, C. A., Mansell, A. S., Brady, G., Brint, E., Dunne, A., Gray, P., Harte, M. T., et al. (2001). Mal (MyD88-adapter-like) is required for Toll-like receptor-4 signal transduction, Nature 413, 78-83.

Galan, J. E., and Curtiss, R., 3rd (1989). Cloning and molecular characterization of genes whose products allow Salmonella typhimurium to penetrate tissue culture cells, Proc Natl Acad Sci USA 86, 6383-7.

Goldring, C. E., Reveneau, S., Pinard, D., and Jeannin, J. F. (1998). Hyporesponsiveness to lipopolysaccharide alters the composition of NF-kappaB binding to the regulatory regions of inducible nitric oxide synthase gene, Eur J Immunol 28, 2960-70.

Greisman, S. E., Young, E. J., and Woodward, W. E. (1966). Mechanisms of endotoxin tolerance. IV. Specificity of the pyrogenic refractory state during continuous intravenous infusions of endotoxin, J Exp Med 124, 983-1000.

Gustafson, G. L., Rhodes, M. J., and Hegel, T. (1995). Monophosphoryl lipid A as a prophylactic for sepsis and septic shock, Prog Clin Biol Res 392, 567-79.

Hagberg, L., Hull, R., Hull, S., McGhee, J. R., Michalek, S. M., and Svanborg Eden, C. (1984). Difference in susceptibility to gram-negative urinary tract infection between C3H/HeJ and C3H/HeN mice, Infect Immun 46, 839-44.

Hayashi, F., Smith, K. D., Ozinsky, A., Hawn, T. R., Yi, E. C., Goodlett, D. R., Eng, J. K., Akira, S., Underhill, D. M., and Aderem, A. (2001). The innate immune response to bacterial flagellin is mediated by Toll-like receptor 5, Nature 410, 1099-103.

Hemmi, H., Takeuchi, O., Kawai, T., Kaisho, T., Sato, S., Sanjo, H., Matsumoto, M., Hoshino, K., Wagner, H., Takeda, K., and Akira, S. (2000). A Toll-like receptor recognizes bacterial DNA, Nature 408, 740-5.

Henricson, B. E., Benjamin, W. R., and Vogel, S. N. (1990). Differential cytokine induction by doses of lipopolysaccharide and monophosphoryl lipid A that result in equivalent early endotoxin tolerance, Infect Immun 58, 2429-37.

Horng, T., Barton, G. M., and Medzhitov, R. (2001). TIRAP: an adapter molecule in the Toll signaling pathway, Nat Immunol 2, 835-41.

Hoshino, K., Takeuchi, O., Kawai, T., Sanjo, H., Ogawa, T., Takeda, Y., Takeda, K., and Akira, S. (1999). Cutting edge: Toll-like receptor 4 (TLR4)-deficient mice are hyporesponsive to lipopolysaccharide: evidence for TLR4 as the Lps gene product, J Immunol 162, 3749-52.

Kaniga, K., Bossio, J. C., and Galan, J. E. (1994). The Salmonella typhimurium invasion genes invF and invG encode homologues of the AraC and PulD family of proteins, Mol Microbiol 13, 555-68.

Kastenbauer, S., and Ziegler-Heitbrock, H. W. (1999). NF-kappaB1 (p50) is upregulated in lipopolysaccharide tolerance and can block tumor necrosis factor gene expression, Infect Immun 67, 1553-9.

Kawai, T., Adachi, O., Ogawa, T., Takeda, K., and Akira, S. (1999). Unresponsiveness of MyD88-deficient mice to endotoxin, Immunity 11, 115-22.

Knop, J., and Martin, M. U. (1999). Effects of IL-1 receptor-associated kinase (IRAK) expression on IL-1 signaling are independent of its kinase activity, FEBS Lett 448,81-5.

Kobayashi, K., Hatano, M., Otaki, M., Ogasawara, T., and Tokuhisa, T. (1999). Expression of a murine homologue of the inhibitor of apoptosis protein is related to cell proliferation, Proc Natl Acad Sci USA 96, 1457-62.

Li, X., Commane, M., Bums, C., Vithalani, K., Cao, Z., and Stark, G. R. (1999). Mutant cells that do not respond to interleukin-1 (IL-1) reveal a novel role for IL-1 receptor-associated kinase, Mol Cell Biol 19, 4643-52.

Maschera, B., Ray, K., Bums, K., and Volpe, F. (1999). Overexpression of an enzymically inactive interleukin-1-receptor- associated kinase activates nuclear factor-kappaB, Biochem J 339, 227-31.

McMahan, C. J., Slack, J. L., Mosley, B., Cosman, D., Lupton, S. D., Brunton, L. L., Grubin, C. E., Wignall, J. M., Jenkins, N. A., Brannan, C. I., and et al. (1991). A novel IL-1 receptor, cloned from B cells by mammalian expression, is expressed in many cell types, Embo J 10, 2821-32.

Medzhitov, R., and Janeway, C. A., Jr. (1997). Innate immunity: the virtues of a nonclonal system of recognition, Cell 91, 295-8.

Medzhitov, R., Preston-Hurlburt, P., Kopp, E., Stadlen, A., Chen, C., Ghosh, S., and Janeway, C. A., Jr. (1998). MyD88 is an adaptor protein in the hToll/IL-1 receptor family signaling pathways, Mol Cell 2, 253-8.

Muzio, M., Bosisio, D., Polentarutti, N., D'Amico, G., Stoppacciaro, A., Mancinelli, R., van't Veer, C., Penton-Rol, G., Ruco, L. P., Allavena, P., and Mantovani, A. (2000). Differential expression and regulation of toll-like receptors (TLR) in human leukocytes: selective expression of TLR3 in dendritic cells, J Immunol 164, 5998-6004.

Muzio, M., Ni, J., Feng, P., and Dixit, V. M. (1997). IRAK (Pelle) family member IRAK-2 and MyD88 as proximal mediators of IL-1 signaling, Science 278, 1612-5.

Nomura, F., Akashi, S., Sakao, Y., Sato, S., Kawai, T., Matsumoto, M., Nakanishi, K., Kimoto, M., Miyake, K., Takeda, K., and Akira, S. (2000). Cutting edge: endotoxin tolerance in mouse peritoneal macrophages correlates with downregulation of surface toll-like receptor 4 expression, J Immunol 164, 3476-9.

Penheiter, K. L., Mathur, N., Giles, D., Fahlen, T., and Jones, B. D. (1997). Non-invasive Salmonella typhimurium mutants are avirulent because of an inability to enter and destroy M cells of ileal Peyer's patches, Mol Microbiol 24, 697-709.

Poltorak, A., He, X., Smirnova, I., Liu, M. Y., Huffel; C. V., Du, X., Birdwell, D., Alejos, E., Silva, M., Galanos, C., et al. (1998). Defective LPS signaling in C3H/HeJ and C57BL/10ScCr mice: mutations in Tlr4 gene, Science 282, 2085-8.

Qureshi, S. T., Lariviere, L., Leveque, G., Clermont, S., Moore, K. J., Gros, P., and Malo, D. (1999). Endotoxin-tolerant mice have mutations in Toll-like receptor 4 (Tlr4), J Exp Med 189, 615-25.

Salkowski, C. A., Detore, G., Franks, A., Falk, M. C., and Vogel, S. N. (1998). Pulmonary and hepatic gene expression following cecal ligation and puncture: monophosphoryl lipid A prophylaxis attenuates sepsis-induced cytokine and chemokine expression and neutrophil infiltration, Infect Immun 66, 3569-78.

Schnare, M., Barton, G. M., Holt, A. C., Takeda, K., Akira, S., and Medzhitov, R. (2001). Toll-like receptors control activation of adaptive immune responses, Nat Immunol 2, 947-50.

Shahin, R. D., Engberg, I., Hagberg, L., and Svanborg Eden, C. (1987). Neutrophil recruitment and bacterial clearance correlated with LPS responsiveness in local gram-negative infection, J Immunol 138, 3475-80.

Swantek, J. L., Tsen, M. F., Cobb, M. H., and Thomas, J. A. (2000). IL-1 receptor-associated kinase modulates host responsiveness to endotoxin, J Immunol 164, 4301-6.

Takeuchi, O., Hoshino, K., Kawai, T., Sanjo, H., Takada, H., Ogawa, T., Takeda, K., and Akira, S. (1999). Differential roles of TLR2 and TLR4 in recognition of gram-negative and gram-positive bacterial cell wall components, Immunity 11, 443-51.

Van Heel, D. A., McGovern, D. P., and Jewell, D. P. (2001). Crohn's disease: genetic susceptibility, bacteria, and innate immunity, Lancet 357, 1902-4.

Wesche, H., Gao, X., Li, X., Kirschning, C. J., Stark, G. R., and Cao, Z. (1999). IRAK-M is a novel member of the Pelle/interleukin-1 receptor-associated kinase (IRAK) family, J Biol Chem 274, 19403-10.

Wesche, H., Henzel, W. J., Shillinglaw, W., Li, S., and Cao, Z. (1997). MyD88: an adapter that recruits IRAK to the IL-1 receptor complex, Immunity 7, 837-47.

Yamin, T. T., and Miller, D. K. (1997). The interleukin-1 receptor-associated kinase is degraded by proteasomes following its phosphorylation, J Biol Chem 272, 21540-7.

Zhang, F. X., Kirschning, C. J., Mancinelli, R., Xu, X. P., Jin, Y., Faure, E., Mantovani, A., Rothe, M., Muzio, M., and Arditi, M. (1999). Bacterial lipopolysaccharide activates nuclear factor-kappaB through interleukin-1 signaling mediators in cultured human dermal endothelial cells and mononuclear phagocytes, J Biol Chem 274, 7611-4.

Ziegler-Heitbrock, H. W. (1995). Molecular mechanism in tolerance to lipopolysaccharide, J Inflamm 45, 13-26.

Ziegler-Heitbrock, H. W., Wedel, A., Schraut, W., Strobel, M., Wendelgass, P., Sternsdorf, T., Bauerle, P. A., Haas, J. G., and Riethmuller, G. (1994). Tolerance to lipopolysaccharide involves mobilization of nuclear factor kappa B with predominance of p50 homodimers, J Biol Chem 269, 17001-4.

We claim:

1. Isolated nucleic acid encoding a murine IRAK-M protein comprising the nucleic acid sequence depicted in SEQ ID NO.: 1.

2. Isolated nucleic acid that encodes a murine IRAK-M protein comprising the amino acid sequence depicted in SEQ ID NO.: 2.

3. Isolated IRAK-M protein encoded by the nucleic acid sequence depicted in SEQ ID NO.: 1.

4. Isolated IRAK-M protein comprising the amino acid sequence depicted in SEQ ID NO.: 2.

5. An expression vector comprising the nucleic acid which has the sequence depicted in SEQ ID NO.: 1.

6. An expression vector comprising nucleic acid encoding the amino acid sequence depicted in SEQ ID NO.: 2.

7. The vector of claim 5 further comprising DNA sufficient for expression of the DNA encoding the amino acid sequence depicted in SEQ ID NO.: 2.

8. A cell transformed with the vector of claim 5.

9. A cell transformed with the vector of claim 6.

10. A cell transformed with the vector of claim 7.

11. A method for producing murine IRAK-M comprising culturing cells that contain a vector comprising DNA encoding murine IRAK-M under conditions appropriate for expression of the DNA, wherein murine IRAK-M is thereby produced.

12. An isolated cell which does not comprise nucleic acid encoding a functional IRAK-M.

13. An isolated IRAK-M^{-/-} cell.

14. The isolated cell of claim 12, wherein the cell is a mammalian cell.

15. The isolated cell of claim 14, wherein the cell is a macrophage.

16. The isolated cell of claim 14, wherein the cell is a mouse or human cell.

17. The isolated cell of claim 12, comprising exogenous nucleic acid encoding IRAK-M.

18. The isolated cell of claim 17, wherein the IRAK-M is murine IRAK-M.

19. The isolated cell of claim 17, wherein the nucleic acid is contained in an expression vector.

20. The isolated cell of claim 12, obtained from an IRAK-M deficient (IRAK^{-M/-}) transgenic non-human animal.

21. The isolated cell of claim 20, wherein the transgenic non-human animal is a mouse.

22. A method of identifying a compound that modulates the innate immune response in an individual, comprising combining cells expressing murine IRAK-M with a candidate compound, and determining whether the candidate compound modulates IRAK-M activity in the cells, wherein modulation of IRAK-M activity in the cells by the candidate compound indicates that the candidate compound modulates the innate immune response in the individual.

23. The method of claim 22, wherein the combining is conducted under conditions appropriate for entry of the candidate compound into the cells.

24. The method of claim 22, further comprising comparing IRAK-M activity in the presence of the candidate compound with the IRAK-M activity for a standard deficient in IRAK-M activity, wherein IRAK-M activity in the presence of the candidate compound which is comparable to IRAK-M activity for the standard indicates that the candidate compound is an IRAK-M inhibitor.

25. The method of claim 24 , wherein the standard is IRAK-M activity of a cell which does not express IRAK-M.

26. The method of claim 22, wherein inhibition of IRAK-M activity in the cells by the candidate compound indicates that the compound inhibits IRAK-M activity, thereby identifying a compound that enhances the innate immune response.

27. The method of claim 26, further comprising the step of comparing the IRAK-M activity in the presence of the compound with IRAK-M activity for a standard known to be deficient in IRAK-M activity, wherein IRAK-M activity in the presence of the compound which is comparable to IRAK-M activity for the known standard indicates that the compound is an IRAK-M inhibitor.

28. The method of claim 27, wherein the standard is IRAK-M activity for a cell which does not express IRAK-M.

29. The method of claim 26, wherein the innate immune response is production of inflammatory cytokines or chemokines.

30. A method of identifying a compound that produces an anti-inflammatory effect and an immunoinhibitory effect in a subject, comprising combining cells expressing IRAK-M with a candidate compound and determining whether the candidate compound enhances IRAK-M activity in the cells, wherein if enhancement of IRAK-M activity occurs in the cells, a candidate compound that produces an anti-inflammatory effect and an immunoinhibitory effect is identified.

31. A method of identifying a compound that produces an immunostimulatory effect in a subject, comprising combining cells expressing IRAK-M with a candidate compound and determining whether the candidate compound inhibits IRAK-M activity in the cells, wherein if inhibition of IRAK-M activity occurs in the cells, a compound that produces an immunostimulatory effect is identified.

32. The method of claim 31, wherein the standard is the IRAK-M activity for a cell which does not express IRAK-M.

33. A method of producing an anti-inflammatory effect and an immunoinhibitory effect in an individual, comprising administering to the individual a compound that enhances IRAK-M in cells in sufficient quantity to enhance IRAK-M, thereby producing an anti-inflammatory effect and an immunoinhibitory effect in the individual.

34. A method of treating an inflammatory condition in an individual comprising administering to the individual a compound that enhances IRAK-M activity in the cells in the individual thereby producing an anti-inflammatory effect in the individual.

35. The method of claim 34, wherein the inflammatory condition is an autoimmune condition.

36. The method of claim 35, wherein the autoimmune condition is rheumatoid arthritis or lupus erythematosis.

37. A method of determining whether a compound is an IRAK-M inhibitor, comprising:
(a) contacting a cell expressing IRAK-M with a candidate compound and measuring the production by the cell of an inflammatory cytokine or chemokine upon stimulation with a TLR or IL-IR ligand;
(b) comparing production by the cell of the inflammatory cytokine or chemokine in (a) with production by the cell of the inflammatory cytokine or chemokine in the absence of the candidate compound;
(c) contacting a cell which does not express IRAK-M with the candidate compound and measuring production by the cell of an inflammatory cytokine or chemokine upon stimulation with a TLR or IL-1R ligand; and
(d) comparing production by the cell of the inflammatory cytokine or chemokine in (c) with production by the cell of the inflammatory cytokine or chemokine in the absence of the candidate compound,
wherein if production in (a) which is more than production in (b), and the production in (c) which is comparable to production in (d) indicates that the compound is an IRAK-M inhibitor.

38. The method of claim 37, wherein the TLR or IL-1R ligand is capable of increasing production of an inflammatory cytokine.

39. A method of determining whether a compound is an IRAK-M inhibitor comprising:
(a) contacting a cell expressing IRAK-M with the candidate compound and measuring production by the cell of an inflammatory cytokine or chemokine upon stimulation with a pathogen;
(b) comparing production by the cell of the inflammatory cytokine or chemokine of step (a) with production by the cell of the inflammatory cytokine or chemokine in the absence of the candidate compound;
(c) contacting a cell which does not express IRAK-M with the candidate compound on a measuring production by the cell of an inflammatory cytokine or chemokine upon stimulation with a pathogen;
(d) comparing production by the cell of the inflammatory cytokine or chemokine in step (c) with production by the cell of the inflammatory cytokine or chemokine in the absence of the candidate compound.
wherein if production in (a) which is more than production in (b), and production in (c) which is comparable to the production in (d) indicates that the compound is an IRAK-M inhibitor.

40. The method of claim 39, wherein the pathogen is Salmonella typhimurium, Escherichia coli or Listeria monocytogenes.

41. The method of claim 37 or 39, wherein the inflammatory cytokine is IL-6, TNFα or IL-12p40.

42. A method of determining whether a compound is an IRAK-M inhibitor comprising:
(a) contacting a cell expressing IRAK-M with the candidate compound and measuring NF-κB activation in the cell;
(b) comparing the NF-κB activation measured in (a) with the activation of NF-κB measured in a cell expressing IRAK-M in the absence of the candidate compound;
(c) contacting a cell which does not express IRAK-M with the candidate compound and measuring the activation of NF-κB in the cell;
(d) comparing the NF-κB activation measured in (c) with the NF-κB activation measured in a cell which does not express IRAK-M in the absence of the candidate compound;
wherein the activation measured in (a) which is more than the activation measured in (b), and the activation measured in (c) which is comparable to the activation measured in (d) indicates that the compound is an IRAK-M inhibitor.

43. The method of claim 42, wherein NF-κB activation is increased upon TLR stimulation.

44. The method of claim 42, wherein NF-κB activation is determined by examining the phosphorylation state of p38, IκBα, ERK1/2 or JNK.

45. The method of claim 42, wherein NF-κB activation is detected by measuring IκBαdegradation.

46. A method of detecting an agonist of IRAK-M activity, comprising:
(a) contacting a cell expressing AK-M with a candidate compound and measuring production of an inflammatory cytokine or chemokine upon stimulation with a TLR or IL-1R ligand; and
(b) comparing production by the cell of an inflammatory cytokine or chemokine in (a) with the production by the cell of the inflammatory cytokine or chemokine in the absence of the candidate compound,
wherein if production in (a) which is less than production in (b) indicates that the compound is an IRAK-M agonist.

47. A method of detecting an agonist of IRAK-M activity, comprising:
(a) contacting a cell expressing IRAK-M with a candidate compound and measuring production of an inflammatory cytokine or chemokine upon stimulation with a pathogen; and
(b) comparing production by the cell of an inflammatory cytokine or chemokine in (a) with the production by the cell of the inflammatory cytokine or chemokine in the absence of the candidate compound,
wherein if production in (a) which is less than production in (b) indicates that the compound is an IRAK-M agonist.

48. A method of determining whether a compound is an IRAK-M agonist comprising:
(a) contacting a cell expressing IRAK-M with the candidate compound and measuring NF-κB activation in the cell; and
(b) comparing the NF-κB activation measured in (a) with the activation of NF-κB measured in a cell expressing IRAK-M in the absence of the candidate compound;
wherein the activation measured in (a) which is less than the activation measured in (b), indicates that the compound is an IRAK-M agonist.
